(19) United States
(12) Patent Application Publication
(10) Pub. No.: US 2026/0241088 A1
(43) Pub. Date: Aug. 20, 2026

(54) Gas Supply Apparatus

(71) Applicant: Spectrum Medical Limited, Gloucester, Gloucestershire (GB)

(72) Inventor: Stephen Turner, Gloucester, Gloucestershire (GB)

(21) Appl. No.: 19/130,790

(22) PCT Filed: Nov. 9, 2023
(86) PCT No.: PCT/GB2023/052934

§ 371 Date: May 16, 2025

(30) Foreign Application Priority Data
Nov. 18, 2022 (GB) 2217323.1
Nov. 18, 2022 (GB) 2217324.9

(51) Int. Cl. A61M 1/16; A61M 39/00; A61M 39/22

(52) U.S. Cl. CPC A61M 1/1698; A61M 39/22; A61M 2039/0009; A61M 2202/0007; A61M 2202/0208; A61M 2202/0413; A61M 2205/3327; A61M 2205/3334; A61M 2205/50

(57) ABSTRACT

An apparatus for controlling a supply of an oxygenation gas to an oxygenator (140) comprises a mixing chamber (161) having an internal volume (165), a plurality of inlets (162a, 162b) and a plurality of outlets (167a, 167b). Each inlet is configured to receive a different supply gas, and each outlet is configured to convey an oxygenation gas from the internal volume to the oxygenator (140). The apparatus further comprises a plurality of inlet valves (164a, 164b) each disposed between a respective inlet and the internal volume of the mixing chamber, and a plurality of outlet valves (166a, 166b) each disposed between the internal volume of the mixing chamber and a respective outlet. The apparatus further comprises a controller (150) configured to open and close each of the inlet valves and each of the outlet valves independently of one another.

The present disclosure relates to an apparatus for controlling a supply of an oxygenation gas to an oxygenator. Embodiments of the apparatus disclosed herein can be used in a cardiac perfusion system.

Cardiac perfusion is a medical procedure involving extracorporeal oxygenation of a patient's blood. Cardiac perfusion is performed, for example, when a patient is unable to oxygenate their own blood by breathing, such as during heart and/or lung surgery. Extracorporeal oxygenation usually uses a pump that acts in place of the patient's own heart and an oxygenator that acts in place of the patient's own lungs. The oxygenator removes carbon dioxide from, and adds oxygen to, the patient's blood. Cardiac perfusion is sometimes known in the art as extracorporeal perfusion or extracorporeal circulation. International Patent Publication No. WO 2019/166823 A1 by the present Applicant discloses an oxygenator for a cardiac system. The oxygenator has a gas inlet zone, which is fluidly connected with a gas-blood interface. The gas inlet zone is separated into two compartments by a partition, causing the gas-blood interface region to be divided into two regions. Each compartment of the gas inlet zone is connected to a respective gas supply, and a flow controller is located between each gas supply and the inlet zone to which it is connected. The flow controllers allow the gas supply to be modulated differently for different interface gas-blood regions.

The multiple gas-blood interface regions in oxygenators such as that disclosed in WO 2019/166823 A1 enable sophisticated ventilation techniques that closely control a patient's blood gas saturation levels. However, putting such ventilation techniques into practice requires each gas-blood interface region to be supplied with the correct gas at the right time. This may require clinicians to manually change the connections between the oxygenator and the gas supply, which can be time-consuming and introduces a risk of human error. The present disclosure aims to overcome or mitigate such difficulties with supplying gases to an oxygenator.

### SUMMARY

A first aspect of the present disclosure relates to an apparatus for controlling a supply of an oxygenation gas to an oxygenator. The apparatus comprises a mixing chamber having an internal volume, a plurality of inlets and a plurality of outlets, wherein each inlet is configured to receive a different supply gas, and each outlet is configured to convey an oxygenation gas from the internal volume to the oxygenator. The apparatus further comprises a plurality of inlet valves, wherein each inlet valve is disposed between a respective inlet and the internal volume of the mixing chamber. The apparatus further comprises a plurality of outlet valves, wherein each outlet valve is disposed between the internal volume of the mixing chamber and a respective outlet. The apparatus further comprises a controller configured to open and close each of the inlet valves and each of the outlet valves independently of one another.

By independently opening and closing each of the inlet and outlet valves, the apparatus disclosed herein can automatically cause the oxygenator to operate in accordance with a different mode of operation, without requiring a clinician to change any connections between the oxygenator and a hospital gas supply. Instead of manually changing such connections, the clinician can instruct the apparatus to change the oxygenator to a different mode of operation (e.g., by selecting an option from a user interface). This reduces the risk of human errors that might arise when manually changing connections between the oxygenator and hospital gas supply, which in turn improves patient safety. Furthermore, by avoiding the need to change any connections manually, the operating mode of the oxygenator can be rapidly changed as clinical needs arise, thereby improving clinical outcomes for patients.

The apparatus disclosed herein also allows the different modes of operation of the oxygenator to be implemented with a single mixing chamber. That is, the disclosed configuration of the mixing chamber with respect to the inlet valves and outlet valves allows all of the oxygenator's modes of operation to be put into practice, without requiring a second mixing chamber or any direct connections between the oxygenator and the hospital gas supply. A ventilation system incorporating the disclosed apparatus thus has a reduced size and complexity.

The term “oxygenation gas” refers to a gas that is provided to an oxygenator by the apparatus. The oxygenation gas is used by the oxygenator to oxygenate blood. Depending on the composition of the oxygenation gas and/or the composition of the blood, oxygenation may include adding oxygen to the blood, removing carbon dioxide from the blood, or both. An oxygenation gas may be pure oxygen, or may comprise oxygen mixed with one or more other gases. For example, an oxygenation gas may comprise a mixture of oxygen with nitrogen and/or carbon dioxide. As another example, an oxygenation gas may comprise a mixture of nitrogen and oxygen (but no carbon dioxide).

The term “supply gas” refers to a component of an oxygenation gas. An oxygenation gas may consist of a single supply gas, or an oxygenation gas may be a mixture of two or more supply gases. The source of a supply gas may be a hospital gas supply. A hospital gas supply (also known as a medical gas supply) is an installation that distributes various gases from a source (e.g. a gas cylinder) to a plurality of outlets located in a hospital or other medical facility. Oxygen and air are examples of supply gases that the apparatus may receive from a hospital gas supply. Alternatively or in addition, the source of a supply gas may be a gas cylinder co-located with the apparatus disclosed herein. As used herein, the term “valve” refers to a component or device that is capable of regulating (i.e., increasing, decreasing and/or stopping) the flow rate of a gas. Each valve may be of any suitable type including, but not limited to, a ball valve, a butterfly valve, a diaphragm valve, a gate valve or a needle valve. In the following disclosure, valves are said to have a “position”, but this is not intended to place any limitations on the type of valve or the manner in which the valves are actuated.

Each valve may have, or may be coupled to, an actuator to allow the controller to open and close the valve. For example, the valve may have an electromechanical actuator, such as a motor or a solenoid, to which the controller can send an electrical signal (e.g., a voltage) to cause the valve to open or close. Other suitable actuators may be used, such as hydraulic or pneumatic actuators.

As used herein, the term “open” refers to any position of a valve in which the valve permits passage of a gas. The term “fully-open” refers to a position of the valve in which passage of gas through the valve is maximal. The term “closed” refers to a position of the valve in which the valve does not permit any passage of a gas (in other words, the valve completely prohibits passage of the gas). The term “partially-open” refers to an intermediate position that is between the fully-open position and the closed position. Hence, a valve can be said to be “open” when it is in the fully-open position, or when it is in the partially-open position.

The term “opening” refers to moving the position of a valve towards the fully-open position, but does not necessarily require the valve to reach the fully-open position. The term “fully-opening” refers to moving the position of a valve such that it reaches the fully-open position. The term “closing” refers to moving the position of the valve towards the closed position, but does not necessarily require the valve to reach the closed position. The term “fully-closing” refers to moving the position of a valve such that it reaches the closed position. The term “adjusting” is used herein to encompass the actions of opening, fully-opening, closing and fully-closing a valve.

The apparatus disclosed herein may control the composition of the oxygenation gas by opening and/or closing the inlet valves, so as to vary the relative proportions of different supply gases entering the mixing chamber. For example, the apparatus may control the concentration of oxygen in the oxygenation gas by opening and/or closing the inlet valves to adjust the relative proportions of nitrogen, carbon dioxide, and/or oxygen in the oxygenation gas. The apparatus disclosed herein may also control the flow rate of the oxygenation gas into the oxygenator by opening and/or closing the inlet valves and/or outlet valves.

Any of the valves mentioned herein may be implemented by a mass flow controller. A mass flow controller is a device comprising a valve, a sensor and a control loop, wherein the control loop is configured to adjust the position of the valve to achieve a desired flow rate. The control loop receives a flow rate measured by the sensor and the desired flow rate as its inputs, and adjusts the position of the valve to minimise the difference the measured flow rate and the desired flow rate. When a valve is implemented by a mass flow controller, the controller of the apparatus can open and/or close the position of the valve by outputting a signal indicative of a desired flow rate to an input of the control loop of the mass flow controller.

In the following examples, it is assumed that a first inlet of the plurality of inlets is connected to a supply of air. For example, the first inlet may be connected to a supply of medical air from a hospital gas supply. The following examples further assume that a second inlet of the plurality of inlets is connected to a supply of oxygen. For example, the second inlet may be connected to a supply of substantially pure oxygen from a hospital gas supply. The following examples further assume that each outlet is connected to a respective inlet of an oxygenator, which may be a multi-region oxygenator.

A multi-region oxygenator is an oxygenator in which the gas-blood interface is separated into two or more gas-blood interface regions, and in which each gas-blood interface region can be independently supplied with an oxygenation gas. The existence of multiple gas-blood interface regions allows fine control over the exchange of gases within the oxygenator, because blood can be exposed to different oxygenation gas conditions in each region. A dual-region oxygenator is a multi-region oxygenator with exactly two gas-blood interface regions.

The plurality of inlet valves may include a first inlet valve and a second inlet valve, and the controller may be configured to adjust the first inlet valve and the second inlet valve to control a concentration of oxygen in the oxygenation gas.

The first inlet valve is disposed between the first inlet and the internal volume of the mixing chamber. As mentioned above, the first inlet is connected to the supply of air. Adjusting the first inlet valve modulates the proportion of air (which is mostly nitrogen) in the oxygenation gas. The second inlet valve is disposed between the second inlet and the internal volume of the mixing chamber. As mentioned above, the second inlet is connected to the supply of oxygen. Adjusting the second inlet valve modulates the proportion of oxygen in the oxygenation gas. Hence, by adjusting the first and second inlet valves, the concentration of oxygen in the oxygenation gas can be controlled to achieve a particular clinical outcome. For example, increasing the concentration of oxygen in the oxygenation gas can promote oxygen uptake by the patient's blood. As another example, increasing the concentration of oxygen in the oxygenation gas can prevent the formation of gaseous microemboli (which are bubbles of gas in the blood, and often have a high nitrogen content). If gaseous microemboli are already present in the patient's blood, increasing the concentration of oxygen can remove the gaseous microemboli or reduce their number and/or size. Conversely, decreasing the concentration of oxygen in the oxygenation gas can reduce the risk of the patient developing hyperoxia.

The term “concentration of oxygen in the oxygenation gas” is sometimes referred to in the art as a “fraction of inspired oxygen” (abbreviated as “FiO2”).

More generally, the controller may be configured to control a composition of the oxygenation gas by adjusting the inlet valves.

For example, the apparatus may comprise a third inlet and a third inlet valve. The third inlet may be connected to a supply of carbon dioxide from a hospital gas supply. The supply of carbon dioxide may be a supply of pure carbon dioxide, or it may be a supply of carbogen. The controller may control a composition of carbon dioxide in the oxygenation gas by adjusting the third inlet valve. For example, the composition of carbon dioxide in the oxygenation gas may be increased to reduce the risk of the patient developing hypocarbia. The apparatus may have more than three inlets and inlet valves.

The controller may be further configured to cause the oxygenator to operate in a first mode by opening all of the outlet valves. In the first mode, a multi-region oxygenator operates in the same manner as a conventional single-region oxygenator. The apparatus automatically reconfigures how the oxygenation gas is supplied to the oxygenator, such that the multi-region oxygenator operates in the same manner as a conventional single-region oxygenator. This is beneficial for clinicians who do not wish to use the sophisticated ventilation techniques supported by a multi-region oxygenator. Such clinicians can simply instruct the apparatus to cause the oxygenator to operate in the first mode (e.g., by selecting an option from a user interface), without the need to modify the perfusion system by inserting a single-region oxygenator or changing how the oxygenator is connected to a hospital gas supply. By avoiding the need to modify the perfusion system or the connection to the hospital gas supply, the risk of human error is reduced and patient safety is thereby improved.

When the oxygenator is operating in the first mode, the oxygenation gas comprises air and may optionally further comprise additional oxygen. The first inlet valve is open (and, optionally, fully-open) such that air can flow from the hospital gas supply into the mixing chamber via the first inlet. The second inlet valve is adjusted to control the amount of oxygen flowing from the hospital gas supply into the mixing chamber via the second inlet. The second inlet valve is closed when the required concentration of oxygen in the oxygenation gas is the same as the concentration of oxygen in air (i.e., about 21%). The second inlet valve can be progressively opened to increase the concentration of oxygen in the oxygenation gas above the concentration of oxygen in air (i.e., above 21%). The oxygenation gas is permitted to pass through all of the outlet valves and into all gas-blood interface regions of a multi-region oxygenator. The apparatus may adjust the inlet valves and/or the outlet valves to achieve a desired flow rate of oxygenation gas into the oxygenator.

Opening all of the outlet valves may comprise opening each outlet valve to a respective position, wherein each position is chosen to divide a volumetric flow rate of the oxygenation gas through the plurality of outlets in accordance with a predetermined ratio. By precisely dividing the total flow rate of an oxygenation gas between different outlets, the apparatus can better reproduce the functionality of a single-region oxygenator using a multi-region oxygenator.

The predetermined ratio may be chosen to be substantially equal to a ratio between sizes of respective gas-blood interface regions of the oxygenator. Each gas-blood interface region of a multi-region oxygenator is thus provided with an amount of oxygen gas that is commensurate with its size (i.e. volume and/or surface area). This ensures that all gas-blood interface regions of the oxygenator are provided with sufficient oxygenation gas to ensure blood is oxygenated effectively. For example, consider an oxygenator with two gas-blood interface regions, wherein a first gas-blood interface region accounts for 40% of the total volume and/or surface area of the oxygenator, and a second gas-blood interface region accounts for the remaining 60% of the total volume and/or surface area of the oxygenator. In this case, the apparatus opens the outlet valves such that 40% of the volumetric flow rate of oxygenation gas is conveyed to the first gas-blood interface region (via a first outlet valve and a first outlet), and 60% of the volumetric flow rate of oxygenation gas is conveyed to the second gas-blood interface region (via a second outlet valve and a second outlet).

The plurality of outlet valves may include a first outlet valve and a second outlet valve. The controller may be configured to cause the oxygenator to operate in a second mode by: adjusting the first outlet valve between a partially-open position and a fully-open position; and adjusting the second outlet valve between a closed position and an open position.

In the second mode, the apparatus uses the full capabilities of a multi-region oxygenator by adjusting each of the outlet valves to provide a respective flow rate of oxygenation gas to each gas-blood interface region of the oxygenator. The flow rate of oxygenation gas to each gas-blood interface region of the oxygenator is independent of (and, therefore, potentially different from) the flow rate of oxygenation gas to all other gas-blood interface regions of the oxygenator. This can allow sophisticated ventilation techniques that prevent hyperoxia, and minimise or eliminate gaseous microemboli, as discussed in more detail below.

The first and second outlets are connected to different gas-blood interface regions of the multi-region oxygenator. Specifically, the first outlet is connected to a first gas-blood interface region of the multi-region oxygenator via the first outlet valve, and the second outlet is connected to a second gas-blood interface region of the multi-region oxygenator via the second outlet valve.

The controller may be configured to cause the oxygenator to adjust the first and second outlet valves by: opening the first outlet valve and fully-closing the second outlet valve; and opening the second outlet valve. When the apparatus causes the oxygenator to operate in the second mode, the first outlet valve is at least partially-open (and may be fully-open) to convey at least a finite amount of oxygenation gas to the oxygenator, whilst the second outlet valve remains closed. If a clinical situation requires even more oxygenation gas to be conveyed to the oxygenator, the second outlet valve can be progressively opened until the required amount of oxygenation gas is being conveyed. The second outlet valve need not reach the fully-open position.

In this manner, the first outlet valve is kept at least partially-open to ensure the first gas-blood interface region of the multi-region oxygenator receives sufficient oxygenation gas to remove carbon dioxide from the patient's blood. If the patient requires more oxygen, the second outlet valve is opened to allow the oxygenation gas to enter the second gas-blood interface regions of the multi-region oxygenator. The position of the second outlet valve can be adjusted (e.g., incrementally opened and/or closed) to maintain the patient's blood oxygen saturation at a desired level. In general, blood oxygen saturation refers to the percentage of oxygenated haemoglobin in the patient's blood relative to the total amount of haemoglobin in the patient's blood.

The controller may be configured to cause the oxygenator to adjust second outlet valve by, after opening the second outlet valve, fully-closing the second outlet valve. To prevent hyperoxia, the second outlet valve can be closed. This prevents the oxygenation gas entering the second gas-blood interface region of the multi-region oxygenator, and thus reduces oxygen uptake by the patient's blood.

The plurality of inlet valves may include a first inlet valve and a second inlet valve, and the controller may be further configured to cause the oxygenator to operate in the second mode by, when the second outlet valve is closed, adjusting the first inlet valve and the second inlet valve to control a concentration of oxygen in the oxygenation gas. As mentioned above, adjusting the first inlet valve modulates the proportion of air in the oxygenation gas, and adjusting the second inlet valve modulates the proportion of oxygen in the oxygenation gas. Thus, when second outlet valve is fully closed, adjusting the first and second inlet valves controls the concentration of oxygen in the oxygenation gas supplied to the first gas-blood interface region of the multi-region oxygenator. In more detail, the concentration of oxygen in the oxygenation gas supplied to the first gas-blood interface regions of the multi-region oxygenator can be modulated between 21% (when the first inlet valve is open and the second inlet valve is closed) and 100% (when the first inlet valve is closed and the second inlet valve is open). Precise control of the oxygen supplied to the first gas-blood interface region of the multi-region oxygenator is thereby achieved, which can prevent hyperoxia.

The plurality of inlet valves may include a first inlet valve and a second inlet valve, and the controller may be further configured to cause the oxygenator to operate in the second mode by: maintaining the first inlet valve in a closed position while the second inlet valve is open and while the first and second outlet valves are open. In this configuration, the apparatus conveys pure oxygen to both the first and second gas-blood interface regions of the multi-region oxygenator. This configuration can be used to increase blood oxygen saturation when the patient has a high metabolic demand for oxygen.

The controller may be further configured to cause the oxygenator to operate in the second mode by adjusting the second outlet valve between a closed position and an open position while the first inlet valve is closed, while the second inlet valve is closed and while the first outlet valve is open. The flow rate of pure oxygen supplied to the second gas-blood interface region of the multi-region oxygenator is controlled by adjusting the second outlet valve while the other valves are in this configuration. Precise control of oxygen uptake by the patient's blood is thereby achieved, which can prevent hyperoxia.

A first inlet of the plurality of inlets may be connected to (or configured to be connected to) a supply of medical air from a hospital gas supply or a gas cylinder. More specifically, the first inlet may be connected to a supply of medical air. As is known to those in the art, medical air is a mixture of nitrogen and oxygen. Medical air typically contains around 79% nitrogen and around 21% oxygen. Medical air may also contain traces of inert gases (such as argon) and water vapour. Configuring the first inlet to be connected to a supply of medical air may include storing a setting that designates the first inlet for connection to the supply of medical air. Hence, the controller knows that, in use, the first inlet will be connected to the supply of medical air. Storing a setting may include setting the value of a parameter in configuration data stored in the controller's memory.

A second inlet of the plurality of inlets may be connected to (or configured to be connected to) a supply of oxygen from a hospital gas supply or a gas cylinder. Configuring the second inlet to be connected to a supply of oxygen may include storing a setting that designates the second inlet for connection to the supply of oxygen. Hence, the controller knows that, in use, the second inlet will be connected to the supply of oxygen.

Each outlet may be connected to (or configured to be connected to) a respective inlet of a multi-region oxygenator. In other words, each outlet of the mixing chamber can be connected to a different inlet of a multi-region oxygenator. This allows the apparatus to switch between the oxygenator's first and second modes of operation, without needing to change any connections between the hospital gas supply, the mixing chamber and the oxygenator. Configuring each outlet to be connected to a respective inlet of a multi-region oxygenator may include storing a setting that designates a first outlet for connection to a first gas-blood interface region of the oxygenator, and designates a second outlet for connection to a second gas-blood interface region of the oxygenator. Hence, the controller knows that, in use, the first outlet will be connected to the first gas-blood interface region and the second outlet will be connected to the second gas-blood interface region.

The plurality of inlets and the plurality of outlets may be in fluid communication with the internal volume of the mixing chamber. In this manner, a plurality of supply gases can enter the internal volume of the mixing chamber, whereupon the supply gases mix to form an oxygenation gas with a substantially uniform composition throughout the internal volume. The oxygenation gas can exit the internal volume of the mixing chamber through the plurality of outlets. Since the composition of the oxygenation gas is substantially uniform throughout the internal volume of the mixing chamber, the oxygenation gas exiting through each outlet has substantially the same composition.

The apparatus may further comprise a pressure sensor configured to measure pressure within the internal volume of the mixing chamber. Measurements of the pressure within the internal volume of the mixing chamber can be used to ensure efficacious operation of the oxygenator and/or to ensure safety, as will now be described.

The controller may be configured to receive a first pressure measurement from the pressure sensor. The controller may be further configured to open and/or close any of the inlet valves and/or any of the outlet valves to minimise a difference between the pressure measurement and a target pressure. The target pressure may be chosen to be greater than the pressure at a gas outlet of the oxygenator. More specifically, the target pressure may be sufficiently greater than the pressure at a gas exhaust of the oxygenator to ensure that there is a pressure differential across the oxygenator, thereby ensuring that the oxygenation gas flows through the oxygenator and allowing gaseous exchange to occur within the oxygenator.

The controller may be configured to receive a second pressure measurement from the pressure sensor. The controller may be further configured to close at least one of the inlet valves when the second pressure measurement exceeds a safety threshold. The ability to close any of all of the inlet valves automatically can be beneficial if the apparatus has a fail-safe mechanism that causes the outlet valves to close when an anomalous situation occurs. The anomalous situation may be, for example, a fault in the perfusion system, an error made by a clinician, or a clinical problem with the patient. The controller may be configured to detect the anomalous situation and automatically close the outlet valves to protect the patient's safety. However, closure of the outlet valves may cause the pressure within the internal volume of the mixing chamber to increase, which may pose a hazard to those working in the vicinity of the mixing chamber. Therefore, the controller can be configured to close at least one of the inlet valves when the pressure inside the mixing within the internal volume of the mixing chamber exceeds a safety threshold, so as to avoid the pressure increasing to an unsafe level.

The apparatus may comprise other types of sensors. For example, and without limitation, the apparatus may comprise: one or more flow sensors, each configured to measure the flow rate of the oxygenation gas through a respective outlet; a temperature configured to measure the temperature of the oxygenation gas in the mixing chamber; an oxygen concentration sensor configured to measure the concentration of oxygen in the oxygenation gas; a carbon dioxide concentration sensor configured to measure the concentration of carbon dioxide in the oxygenation gas; or any combination thereof.

The controller may be configured to control a flow rate of the oxygenation gas through at least one of the outlet valves by adjusting an inlet valve and/or adjusting the at least one outlet valve.

The mixing chamber, the plurality of inlet valves and the plurality of outlet valves may be integrated within a gas blender. In other words, the mixing chamber, inlet valves and outlet valves are supplied as an integrated device, referred to herein as a gas blender. This simplifies the task of connecting the mixing chamber, inlet valves and outlet valves between the gas supply and the oxygenator. A clinician simply needs to connect a tube between each inlet of the gas blender and the correct gas supply, and to connect a tube between each outlet of the gas blender and the correct oxygenator inlet. The clinician does not need to make any other connections between the gas supply and the oxygenator, and does not need to change any connections during the course of a cardiac perfusion procedure.

The controller may be configured to open the first outlet valve to maintain a flow of oxygenation gas through a first outlet to a first gas-blood interface region of the oxygenator, while adjusting the second outlet valve between a fully-closed position and an open position to control a flow of oxygenation gas through a second outlet to a second gas-blood interface region of the oxygenator, wherein the first gas-blood interface region of the oxygenator is smaller than the second gas-blood interface region of the oxygenator. The controller may be further configured to, in response to determining that the flow of oxygenation gas through the first outlet exceeds a first threshold, open the second outlet valve to maintain a flow of oxygenation gas through the second outlet to the first gas-blood interface region while adjusting the first outlet valve between a fully-closed position and an open position to control a flow of oxygenation gas through the first outlet to the first gas-blood interface.

Initially, the first (i.e., smaller) gas-blood interface region used to remove carbon dioxide from the blood while the second (i.e., larger) gas-blood interface region is used to regulate blood oxygen saturation. If the first gas-blood interface region is not large enough to remove carbon dioxide effectively from the patient's blood, which will indicated by a high flow rate of oxygenation gas through the first outlet, the controller automatically switches to using the second (i.e., larger) gas-blood interface region to remove carbon dioxide from the blood. This ensures effective removal of carbon dioxide from the patient's blood.

The controller may be further configured to, in response to determining that the flow of oxygenation gas through the second outlet is below a second threshold, open the first outlet valve to maintain a flow of oxygenation gas through the first outlet to the first gas-blood interface region while adjusting the second outlet valve between the fully-closed position and the open position to control the flow of oxygenation gas through the second outlet to the second gas-blood interface region.

The controller may store a setting that indicates the respective sizes of the first and second gas-blood interface regions. As previously mentioned, the controller may store a setting that designates a first outlet for connection to a first gas-blood interface region of the oxygenator, and designates a second outlet for connection to a second gas-blood interface region of the oxygenator. These settings inform the controller as to which outlet valve controls the flow of oxygenation gas to the larger gas-blood interface region, and which outlet valve controls the flow of oxygenation gas to the smaller gas-blood interface region.

In accordance with a further aspect of the present disclosure, a cardiac perfusion system comprises an apparatus for controlling a supply of an oxygenation gas to an oxygenator as described above. The cardiac perfusion system may further comprise an oxygenator having a plurality of gas-blood interface regions and plurality of oxygenator gas inlets. Each oxygenator gas inlet may be configured to receive an oxygenation gas and to convey the oxygenation gas to a respective gas-blood interface regions of the plurality of gas-blood interface regions. Each outlet of the mixing chamber may be connected to a respective oxygenator gas inlet.

As used herein, “cardiac perfusion” refers to a medical procedure in which blood is removed from a patient, oxygenated, and returned to the patient. Cardiac perfusion encompasses extracorporeal membrane oxygenation (ECMO) and cardiopulmonary bypass (CPB).

A first inlet of the plurality of inlets of the mixing chamber may be connected to a supply of medical air from a hospital gas supply or a gas cylinder. A second inlet of the plurality of inlets of the mixing chamber may be connected to a supply of oxygen from a hospital gas supply or a gas cylinder.

In accordance with a further aspect of the disclosure, methods of controlling an oxygenator are provided. The methods may be performed by a controller which is coupled to a mixing chamber. Upon performing the method, the controller opens and closes a plurality of inlet valves and a plurality of outlet valves of the mixing chamber, so as to cause the oxygenator to operate in accordance with the first and/or second modes of operation disclosed herein.

In accordance with a further aspect of the disclosure, a processor-readable medium is provided. The processor-readable medium comprises processor-executable instructions which, when executed by a processor, cause a controller comprising the processor to perform any of the methods of controlling an oxygenator disclosed herein. The processor-readable medium may be non-transitory (such as a disc or a memory device) or may be transitory (such as a signal).

In accordance with a further aspect of the disclosure, a computer program is provided. The computer program comprises processor-executable instructions which, when executed by a processor, cause a controller comprising the processor to perform any of the methods of controlling an oxygenator disclosed herein.

### BRIEF DESCRIPTION OF THE DRAWINGS

Embodiments will now be described, purely by way of example, with reference to the accompanying drawings, in which like features are denoted by like reference signs, and in which:

FIG. 1 is a schematic diagram of a cardiac perfusion system that includes a gas blender and controller in accordance with the present disclosure;

FIG. 2 is a schematic diagram of the gas blender and oxygenator shown in FIG. 1;

FIG. 3 is a schematic diagram of the controller shown in FIGS. 1 and 2;

FIG. 4 is a schematic diagram illustrating the oxygenator of FIG. 2 when operating in a first mode;

FIG. 5 is a flow chart of a method of causing an oxygenator to operate in the first mode shown in FIG. 4;

FIG. 6 is a schematic diagram illustrating states of the oxygenator of FIG. 2 when operating in a second mode;

FIG. 7 is a flow chart of a method of causing an oxygenator to operate in the second mode shown in FIG. 6; and

FIG. 8 is a flow chart of a method of using different gas-blood interface regions of an oxygenator as a sweep chamber.

### DETAILED DESCRIPTION

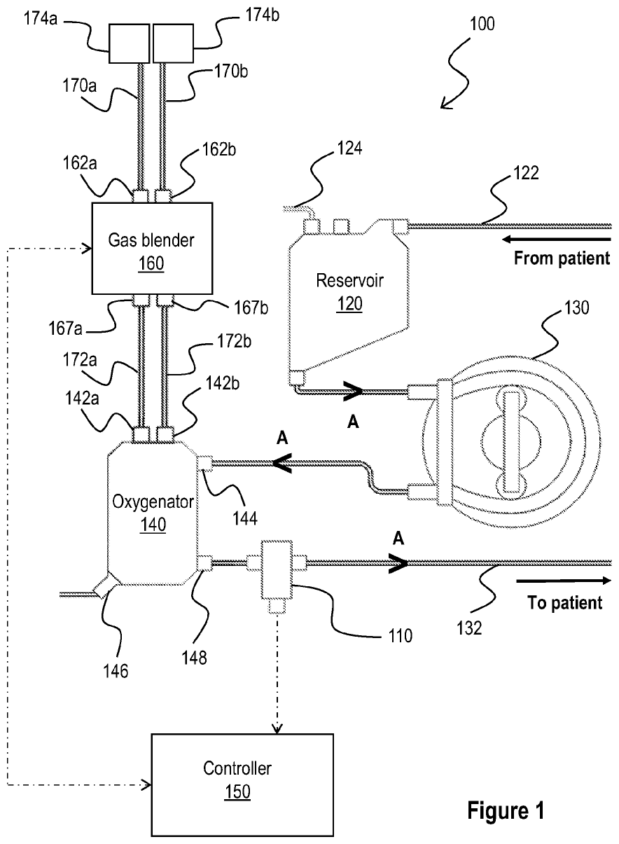

FIG. 1 is a schematic diagram of an example of a cardiac perfusion system 100 incorporating a gas blender 160 and a controller 150 in accordance with the present disclosure. In addition to the gas blender 160 and the controller 150, the cardiac perfusion system 100 comprises one or more sensors 110, a venous reservoir 120, a pump 130 and an oxygenator 140. In use, the cardiac perfusion system 100 receives deoxygenated blood from a patient via a venous line 122, and stores the deoxygenated blood in the venous reservoir 120. The pump 130 pumps deoxygenated blood from the venous reservoir 120, through the oxygenator 140, and returns oxygenated blood to the patient via an arterial line 132 (as shown by the arrows labelled “A”).

The venous reservoir 120 receives deoxygenated blood from the patient via the venous line 122. The venous reservoir 120 may additionally be configured to receive blood from the patient via one or more salvage lines, one or more purge lines, and/or one or more lines configured to carry surgical fluids (e.g. priming solutions, volume expanders, blood, and/or drugs), as represented by line 124 in FIG. 1. The venous reservoir 120 is positioned upstream of the oxygenator 140, between the patient and the oxygenator 140. It will be appreciated that, in other implementations, the exact arrangement of the venous reservoir 120 and the pump 130 may vary. In fact, the system 100 need not necessarily comprise the venous reservoir 120 and the pump 130. In such a case, the oxygenator 140 may be configured to receive the blood directly from the patient.

The pump 130 drives blood through the cardiac perfusion system 100. As shown in FIG. 1, the pump 130 is located downstream of the venous reservoir 120 and upstream of the oxygenator 140. In the depicted embodiment, pump 130 is a roller (or peristaltic) pump. However, it will be appreciated that, depending on the circumstance, other types of pump may be used, such as a centrifugal pump.

The oxygenator 140 receives deoxygenated blood via a blood inlet 144. The oxygenator also receives an oxygenation gas from the gas blender 160 via two or more (in this case, two) oxygenator gas inlets 142a, 142b. The oxygenation gas can include air, oxygen and/or a mixture of air and oxygen, as discussed in more detail below. Blood passes through the oxygenator 140, whereupon gases dissolved in the blood are exchanged with gases received via the oxygenator gas inlets 142a, 142b. The gaseous exchange within the oxygenator 140 generally involves removal of carbon dioxide from the blood, and addition of oxygen to the blood. In this manner, the oxygenator 140 converts deoxygenated blood to oxygenated blood. Oxygenated blood leaves the oxygenator 140 via a blood outlet 148, whereupon it is returned to the patient via the arterial line 132. Waste gas leaves the oxygenator 140 via a gas exhaust 146.

The gas blender 160 receives one or more supply gases and produces an oxygenation gas for supplying to the oxygenator 140. The gas blender 160 comprises two or more (in this case, two) inlets 162a, 162b, and two or more (in this case, two) outlets 167a, 167b. In use, a first inlet 162a is connected to a first source 174a of a first supply gas via a tube 170a. Similarly, in use, a second inlet 162b is connected to a second source 174b of a second supply gas via a tube 170b. Either of the first and second sources 174a, 174b may be a hospital gas supply or a gas cylinder that is not part of a hospital gas supply. In general, the first supply gas is different from the second supply gas. For example, the first supply gas may be medical air, and the second supply gas may be pure oxygen. The gas blender 160 may comprise additional inlets (not shown) for receiving further supply gases. For example, the gas blender 160 may comprise another inlet that, in use, is connected to a source of carbon dioxide. Alternatively or in addition, the gas blender 160 may comprise another inlet that, in use, is connected to a source of an anaesthetic gas (e.g., nitrous oxide). The supply gases may additionally or alternatively comprise other gases common in the art (e.g. helium and/or argon). The gas blender 160 is configured to blend (in other words, mix) the supply gases received via the inlets 162a, 162b to produce an oxygenation gas. The controller 150 controls the gas blender 160 to adjust the composition of the oxygenation gas, as described in more detail below. At times, the oxygenation gas may consist solely of one of the supply gases (e.g., the gas blender 160 may produce an oxygenation gas that consists only of oxygen, or it may produce an oxygenation gas that consists only of medical air). The oxygenation gas can leave the gas blender 160 via the outlets 167a, 167b. In use, a first outlet 167a of the gas blender 160 is connected to a first inlet 142a of the oxygenator 140 via a tube 172a. Similarly, a second outlet 167b of the gas blender 160 is connected to a second inlet 142b of the oxygenator 140 via a tube 172b. The inlets 162a, 162b and outlets 167a, 167b may be ports to which tubes 170a, 170b, 172a, 172b can be connected. The gas blender 160 may comprise additional outlets (not shown) to allow it to be connected to an oxygenator 140 with more than two inlets. That is, the gas blender 160 may have a number of outlets 167a, 167b equal to the number of inlets 142a, 142b of the oxygenator 140.

It is advantageous for number of outlets 167a, 167b from the gas blender 160 to be equal to the number of inlets 142a, 142b to the oxygenator 140, since this allows the supply of oxygenation gas to each gas-blood interface region of the oxygenator 140 to be independent of that to other gas-blood interface regions. However, in some implementations the number of outlets 167a, 167b from the gas blender 160 may be different from the number of inlets 142a, 142b to the oxygenator 140. In such implementations, “Y” connectors may be used to split gas lines and/or join gas lines together. Although the number of inlets 162a, 162b to the gas blender 160 is equal to number of outlets 167a, 167b from the gas blender 160 in the examples shown herein, it should be appreciated that the number of inlets 162a, 162b to the gas blender 160 could be different from number of outlets 167a, 167b from the gas blender 160.

The controller 150 controls the gas blender 160. In more detail, the controller 150 controls the composition of the oxygenation gas produced by the gas blender 160, controls which outlets 167a, 167b of the gas blender 160 are used to convey the oxygenation gas to the oxygenator 140, and controls the flow rate of the oxygenation gas to the oxygenator 140.

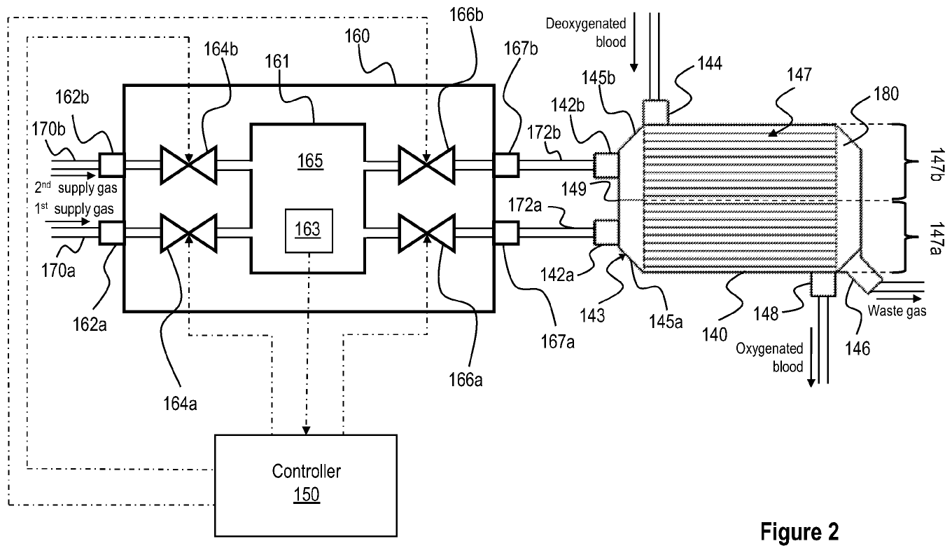

The controller 150 may be a dedicated controller whose only purpose is to control the gas blender 160. Alternatively, the controller 150 may control other components of the perfusion system 100. For example, the controller 150 may control: the pumping of blood by the pump 130; the operation of the oxygenator 140; and/or various valves and/or actuators not illustrated in FIGS. 1 and 2. The controller 150 may also monitor physiological parameters of the patient and/or parameters of the cardiac perfusion system 100 to ensure that a cardiac perfusion procedure takes place safely and efficaciously.

The controller 150 is communicatively connected to the gas blender 160 and the one or more sensors 110, as depicted by the dash-dot lines in FIG. 1. That is, the controller 150 may be configured to communicate with the gas blender 160 and the one or more sensors 110. This communication may occur via hardware connections (e.g. wired connections) or via wireless communication. In this regard, it will be understood that the dash-dot lines depicted in FIG. 1 are merely illustrative and do not necessarily represent physical connections between components. Furthermore, it will be understood that the controller 150 may be communicatively connected to more or fewer components in system 100. For example, the controller 150 may not necessarily communicate directly with the one or more sensors 110, but may instead be configured to communicate with an intermediate transceiver that relays measurements from the sensor 110 to the controller 150. As used herein, “communication” with the controller 150 refers to both the reception of data (e.g. measurements) by the controller 150 and the transmission of data (e.g. instructions or commands) by the controller 150.

The one or more sensors 110 are configured to measure parameters associated with the cardiac perfusion system 100 and/or parameters associated with the patient. Although FIG. 1 shows a single sensor 110 positioned on the arterial line 132, it should be appreciated that this only illustrative. A sensor 110 could be positioned in, or on, any or all of: the venous reservoir 120; the pump 130; the oxygenator 140; the gas blender 160; a blood line (e.g., venous line 122, arterial line 132 and/or any of the intermediate blood lines between the venous reservoir 120 and the oxygenator 140); a gas line (e.g., any of the tubes supplying gas to, or removing gas from, the gas blender 160 and/or the oxygenator 140). Alternatively or in addition, a sensor 110 could be positioned in, or on, the patient. The location of the sensor 110 is chosen in accordance with the parameter that is to be measured. A sensor 110 may be configured to measure, for example: a gas pressure; a gas flow rate; a gas composition (e.g., a partial pressure of oxygen, carbon dioxide or any other gas); a blood pressure; a blood flow rate; a blood composition (e.g., a saturation of oxygen or carbon dioxide in the blood); or a blood volume (e.g., a volume of blood in the venous reservoir 120).

The cardiac perfusion system 100 may optionally further comprise other components not shown in FIG. 1. For example, the cardiac perfusion system 100 may comprise any or all of: a cardioplegia device to stop the patient's heart; a heater-cooler device to regulate the temperature of blood; one or more additional pumps to cause blood to flow from the patient, through the cardiac perfusion system 100 and/or to the patient; and one or more valves to control the flow of blood.

FIG. 2 shows the gas blender 160 and oxygenator 140 in more detail. The gas blender 160 comprises a mixing chamber 161, which has an internal volume 165. The internal volume 165 of the mixing chamber 161 is in fluid communication with the inlets 162a, 162b and the outlets 167a, 167b. An inlet valve 164a, 164b is disposed between each inlet 162a, 162b and the internal volume 165 of the mixing chamber 161. Opening a first inlet valve 164a allows a first supply gas to enter the mixing chamber 161 via the first inlet 162a. Similarly, opening a second inlet valve 164b allows a second supply gas to enter the mixing chamber 161 via the second inlet 162b. Upon entering the internal volume 165 of the mixing chamber 161, the first and second supply gases mix to produce an oxygenation gas. An outlet valve 166a, 166b is disposed between the internal volume 165 of the mixing chamber 161 and each outlet 167a, 167b. Opening a first outlet valve 166a allows the oxygenation gas to leave the internal volume 165 of the mixing chamber 161 via the first outlet 167a. Similarly, opening a second outlet valve 166b the oxygenation gas to leave the internal volume 165 of the mixing chamber 161 via the second outlet 167b. The composition of the oxygenation gas is substantially uniform throughout the mixing chamber 161 and, therefore, the composition of the oxygenation gas leaving the mixing chamber 161 via the first outlet 167a is substantially the same as the composition of the oxygenation gas leaving the mixing chamber 161 via the second outlet 167b.

The controller 150 is configured to communicate a respective control signal to each of the inlet valves 164a, 164b and to each of the outlet valves 166a, 166b. Each control signal is configured to cause a respective valve 164a, 164b, 166a, 166b to open or close. The controller 150 can cause each of the inlet valves 164a, 164b and each of the outlet valves 166a, 166b to open and close independently of one another. By independently controlling each of the valves 164a, 164b, 166a, 166b, the controller 150 can perform various tasks including switching between operating modes of a multi-region oxygenator 140, controlling the composition of the oxygenation gas, and controlling the flow rate of the oxygenation gas to the oxygenator 140. In more detail, the controller 150 can control the composition of the oxygenation gas by opening or closing each inlet valve 164a, 164b to allow supply gases to enter the mixing chamber 161 in a desired ratio. The controller can control the flow rate of the oxygenation gas to the oxygenator 140 by opening or closing each valve 164a, 164b, 166a, 166b to produce a desired throughput of gas from the inlets 162a, 162b to the outlets 167a, 167b.

The mixing chamber 161 may comprise a pressure sensor 163 within its internal volume 165. The pressure sensor 163 is configured to measure the pressure of the oxygenation gas within the internal volume 165 of the mixing chamber 161. The controller 150 is configured to receive pressure measurements from the pressure sensor 163, and to control the pressure of the oxygenation gas in the mixing chamber 161 based on the pressure measurements.

For example, the controller 150 may be configured to maintain the pressure in the mixing chamber 161 at a target pressure that ensures the mixing chamber 161 is at a positive pressure with respect to the gas exhaust 146 of the oxygenator 140. In other words, the target pressure may be sufficiently greater than the pressure at the gas exhaust 146 of the oxygenator 140 to ensure that there is a pressure differential across the oxygenator 140, thereby ensuring that the oxygenation gas flows through the oxygenator 140 and allowing gaseous exchange to occur within the oxygenator 140. If a pressure measurement made by the pressure sensor 163 is below the target pressure, the controller 150 may increase the pressure within the internal volume 165 of the mixing chamber 161 by opening any or all of the inlet valves 164a, 164b and/or by closing any or all of the outlet valves 166a, 166b. Conversely, if the pressure measurement is above the target pressure, the controller 150 may decrease the pressure within the internal volume 165 of the mixing chamber 161 by closing any or all of the inlet valves 164a, 164b and/or by opening any or all of the outlet valves 166a, 166b.

As another example, the controller 150 may be configured to prevent the pressure in the mixing chamber 161 exceeding a safe working pressure, referred to herein as a “safety threshold”. If a pressure measurement made by the pressure sensor 163 exceeds the safety threshold, the controller 150 can close some of all of the inlet valves 164a, 164b to prevent further pressure build-up within the mixing chamber 161. Alternatively or additionally, if a pressure measurement made by the pressure sensor 163 exceeds the safety threshold, the controller 150 can open an exhaust valve (not shown in the Figures) to release oxygenation gas from the mixing chamber 161 into the atmosphere or into a gas capture device, and thereby reduce the pressure in the mixing chamber 161.

The oxygenator 140 is a dual-region oxygenator. That is, the oxygenator 140 has a gas-blood interface 147 which is separated into two gas-blood interface regions 147a, 147b, and each gas-blood interface region can be independently supplied with an oxygenation gas via a respective oxygenator gas inlet 142a, 142b. The gas-blood interface 147 is a medium in which gaseous exchange between the oxygenation gas and the blood takes place. As described above, the oxygenator 140 comprises a blood inlet 144 for receiving blood from the patient, and a blood outlet 148 for returning blood to the patient. The oxygenator 140 comprises a first oxygenator gas inlet 142a and a second oxygenator gas inlet 142b. The first oxygenator gas inlet 142a and the second oxygenator gas inlet 142b are each fluidly connected to a gas inlet zone 143. The oxygenator 140 further comprises a gas exhaust 146 for releasing waste gas from the oxygenator 140. The waste gas exits the oxygenator 140 from the gas exhaust 146 via a gas outlet zone 180. The gas exhaust 146 may be coupled to a vacuum pump to promote the flow of oxygenation gas through the oxygenator 140 and/or to assist in extracting waste gas from the oxygenator 140.

Gas inlet zone 143 comprises a partition 149 that divides the gas inlet zone 143 into a plurality of (in this case, two) gas inlet regions 145a, 145b. Each gas inlet region 145a, 145b is configured to receive an oxygenation gas from a respective oxygenator gas inlet 142a, 142b. More specifically, a first gas inlet region 145a is configured to receive oxygenation gas from the first oxygenator gas inlet 142a, and the second gas inlet region 145b is configured to receive oxygenation gas from the second oxygenator gas inlet 142b. Moreover, the first gas inlet region 145a is configured to receive oxygenation gas from the first outlet 167a of the gas blender 160 via tube 172a and the first oxygenator gas inlet 142a. The second gas inlet region 145b is configured to receive oxygenation gas from the second outlet 167b of the gas blender 160 via tube 172b and the second oxygenator gas inlet 142b.

The gas inlet zone 143 is fluidly connected with the gas-blood interface 147. The gas outlet zone 180 is fluidly connected with the gas-blood interface 147. Hence, the oxygenation gas enters the gas-blood interface 147 from the gas inlet zone 143 and exits the gas-blood interface 147 via the gas outlet zone 180. The gas-blood interface 147 may comprise one or more hollow fibre groups, each hollow fibre group comprising a plurality of hollow fibres. Each hollow fibre group may comprise inlet potting in fluid connection with the gas inlet zone 143. Each hollow fibre group may comprise outlet potting in fluid connection with the gas outlet zone 180.

The gas-blood interface 140 is configured to be supplied with the oxygenation gas to expose the blood to oxygen. For example, the oxygenation gas may enter the hollow fibre groups via the inlet potting from the gas inlet zone 143. The blood enters the oxygenator 140 via the blood inlet 144. The blood and the oxygenation gas pass through the gas-blood interface 147 as they pass through the oxygenator 140. The gas-blood interface 147 is configured to permit gaseous exchange between the blood and the oxygenation gas. This includes the transfer of gases (e.g. oxygen, carbon dioxide, nitrogen) out of the blood and into the oxygenation gas, as well as the transfer of gases (e.g. oxygen, carbon dioxide) into the blood and out of the oxygenation gas. Following gaseous exchange, the blood (now oxygenated) exits the oxygenator 140, towards the patient, via blood outlet 148 and the oxygenation gas (now waste gas) exits the oxygenator 140 via gas outlet 146.

As mentioned above, the partition 149 divides the gas inlet zone 143 into a first gas inlet region 145a and a second gas inlet region 145b. This, in turn, divides the gas-blood interface 147 into two gas-blood interface regions 147a, 147b, and allows each region to be independently supplied with the oxygenation gas via a respective gas inlet region 145a, 145b. This allows a different volumetric flow rate of the oxygenation gas to be supplied to each region 147a, 147b of the gas-blood interface 147.

The partition 149 shown in FIG. 2 extends through the gas inlet zone 143, but does not extend through the gas-blood interface 147. In this case, the partition 149 may abut the inlet potting of the hollow fibre group to prevent gas flow between the gas-blood interface regions 147a, 147b. Alternatively, the partition 149 need not abut the inlet potting if some degree of gas leakage between the gas inlet regions 145a, 145b is permissible. In other examples, the partition 149 may extend through the gas inlet zone 143 and at least partially through the gas-blood interface 147 to physically separate the gas-blood interface 147 into gas-blood interface regions 147a, 147b.

In the example shown in FIG. 2, the partition 149 divides the gas inlet zone 143 into a first gas inlet region 145a and a second gas inlet region 145b that are of equal size. That is, the partition 149 divides the gas inlet zone 143 (and thus the gas-blood interface 147) in half, such that each of the first gas inlet region 145a and the second gas inlet region 145b comprises 50% of the gas inlet zone 143. The partition 149 thus acts to divide the gas-blood interface 147 in half such that each of the first gas-blood interface region 147a and the second gas-blood interface region 147b comprises 50% of the gas-blood interface 147. However, it will be appreciated that this division is merely an example and the partition 149 (and/or multiple partitions) may be located at various positions in order to create different splits of the gas inlet zone 143 and of the gas-blood interface 147. For example, the partition 149 may be positioned such that the first gas inlet region 145a comprises 40% of the gas inlet zone 143 and the second gas inlet region 145b comprises 60% of the gas inlet zone 143. That is, the gas-blood interface 147 may be divided such that the first gas-blood interface region 147a comprises 40% of the gas-blood interface 147 and the second gas-blood interface region 147b comprises 60% of the gas-blood interface 147.

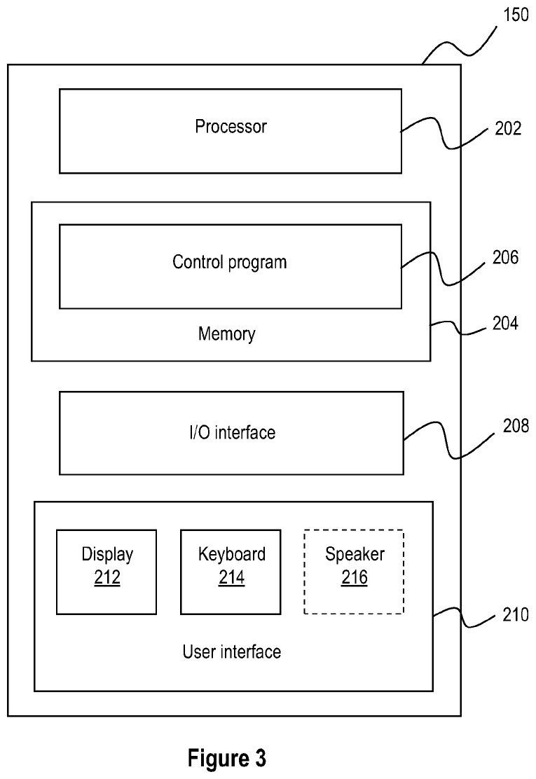

FIG. 3 is a schematic diagram of the controller 150 shown in FIGS. 1 and 2. The controller comprises a processor 202, a memory 204, an input/output (I/O) interface 208 and a user interface 210.

The processor 202 can be any suitable type of data processing device, such as a microprocessor, microcontroller or application specific integrated circuit (ASIC). The processor 202 is communicatively coupled to the memory 204. The memory 204 can include a volatile memory, a non-volatile memory, or both volatile and non-volatile memories. The memory 204 stores a control program 206. The control program 206 includes processor-executable instructions that, when executed by the processor 202, cause the controller 150 to perform any of the methods described below with reference to FIGS. 5, 7 and 8.

The I/O interface 208 receives data from the one or more sensors 110 and outputs control signals. More specifically, the I/O interface 208 communicates control signals to the gas blender 160, which control signals cause the gas blender 160 to operate in the manner disclosed herein. In implementations in which the controller 150 controls other components of the perfusion system 100 in addition to the gas blender 160, the I/O interface 208 may also communicate control signals to the pump 130, the oxygenator 140 and/or other valves and/or actuators not illustrated in FIGS. 1 and 2. The I/O interface 208 may be configured to receive analogue and/or digital data from the one or more sensors 110. Similarly, the I/O interface 208 may be configured to send analogue and/or digital control signals to the gas blender 160 and/or other components of the perfusion system 100.

The user interface 210 comprises a display 212, a keyboard 214 and, optionally, a speaker 216. The display 212 is configured to output a visual indication of information relevant to a cardiac perfusion procedure, such as a visible alert signal and/or a visible indication of particular data collected by the control program 206. The display 212 can be any suitable type of output device. For example, the display 212 may be a liquid crystal display (LCD) screen or an organic light-emitting diode (OLED) screen. The keyboard 214 comprises a plurality of buttons, which a user can use to input information for use by the control program 206. For example, a clinician (such as a perfusionist) can use the keyboard 214 to select: a particular mode of operation of the oxygenator 140; a target blood oxygen saturation to be achieved by the oxygenator 140; a target blood carbon dioxide pressure to be achieved by the oxygenator 140; a composition of the oxygenation gas to be produced by the gas blender 160 (e.g., by setting a desired proportion of oxygen, nitrogen and/or carbon dioxide in the oxygenation gas); and/or a flow rate of oxygenation gas through any or all of the outlets 167a, 167b of the gas blender 160. The display 212 and keyboard 214 may be integrated with one another in the form of a touchscreen. The speaker 216 is capable of outputting audio information, such as an audible alert signal and/or a spoken indication of particular data collected by the monitoring program.

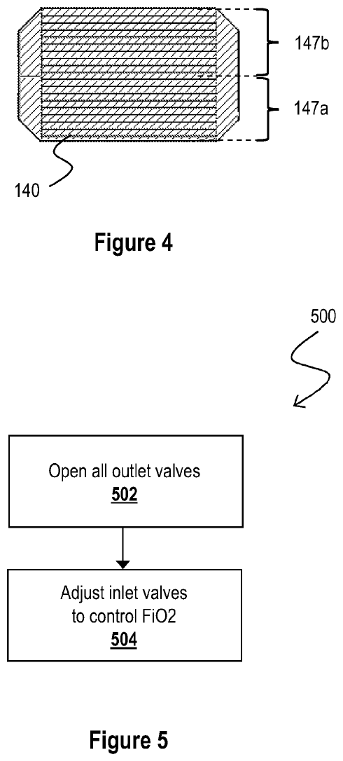

The operation of the gas blender 160 will now be described with reference to FIGS. 4 to 8. The gas blender 160 described herein can automatically cause a multi-region oxygenator 140 (such as the dual-chamber oxygenator described above) to switch between different modes of operation, without requiring a clinician to change any connections between the oxygenator and a supply gas source (e.g., a hospital gas supply).

A first mode of operation of the oxygenator 140 is shown in FIG. 4. In the first mode of operation, the gas blender 160 causes the multi-region oxygenator 140 to operate as if it were a conventional single-region oxygenator. That is, the gas blender 160 conveys oxygenation gas to all gas-blood interface regions 147a, 147b at the same time. The hatched lines in FIG. 4 indicate that the oxygenation gas is conveyed to both the first gas-blood interface region 147a and the second gas-blood interface region 147b. All gas-blood interface regions 147a, 147b receive oxygenation gas with substantially the same composition because the oxygenation gas is mixed within the mixing chamber 161 before being conveyed to the oxygenator 140.

A method 500 of causing the oxygenator to operate in the first mode is illustrated in FIG. 5. The method 500 may begin at operation 502, at which the controller 150 opens all of the outlet valves 166a, 166b of the gas blender 160. Opening all of the outlet valves 166a, 166b allows an oxygenation gas to flow from the gas blender 160 and into all gas-blood interface regions 147a, 147b of the oxygenator 140.

Optionally, operation 502 may include the controller 150 opening the outlet valves 166a, 166b so as to divide the volumetric flow rate of the oxygenation gas through each outlet 167a, 167b in accordance with a predetermined ratio. For example, consider an oxygenator 140 in which the first gas-blood interface region 147a comprises 40% of the gas-blood interface 147 and the second gas-blood interface region 147b comprises 60% of the gas-blood interface 147. The controller 150 may open the first outlet valve 166a less than the second outlet valve 166b, so that 40% of the volumetric flow rate of oxygenation gas leaving the gas blender 160 passes through the first outlet 167a and into the first gas-blood interface region 147a, whilst 60% of the volumetric flow rate of oxygenation gas leaving the gas blender 160 passes through the second outlet 167b and into the second gas-blood interface region 147b. In this manner, each gas-blood interface region 147a, 147b receives an amount of oxygenation gas that is commensurate with its size (e.g., volume or surface area), which improves the ability of the multi-region oxygenator 140 to operate as if it were a conventional single-region oxygenator. Dividing the flow of oxygenation gas through the outlets 167a, 167b in accordance with the size of the gas-blood interface region 147a, 147b also helps to control the rate at which carbon dioxide is removed from the patient's blood. As another example, if the first and second gas-blood interface regions 147a, 147b have equal sizes, the controller 150 may open each outlet valve 166a, 166b equally so as to divide the volumetric flow rate of oxygenation gas equally between all gas-blood interface regions 147a, 147b. It should be appreciated that these are just examples of how the controller 150 may divide the flow of oxygenation gas between the gas-blood interface regions 147a, 147b, and the controller 150 may divide the volumetric flow rate of the oxygenation gas through each outlet 167a, 167b in accordance with other predetermined ratios to achieve a particular clinical outcome.

In order to divide the flow volumetric flow rate appropriately, the controller 150 may access a stored mapping (e.g., a look-up table) of positions of each outlet valve 166a, 166b that cause the volumetric flow rate of the oxygenation gas through each outlet 167a, 167b to be in accordance with the predetermined ratio. Alternatively, the controller 150 may receive a measurement of the volumetric flow rate of the oxygenation gas through each outlet 167a, 167b from one or more gas flow sensors, and may implement a feedback loop to ensure that the volumetric flow rate through each outlet 167a, 167b is in accordance with the predetermined ratio. The gas flow sensors may be situated in any appropriate location, such as: in, or adjacent, an outlet valve 166a, 166b; in, or adjacent, an outlet 167a, 167b of the gas blender 160; in line with the tubes 172a, 172b; and/or in, or adjacent, an inlet 142a, 142b of the oxygenator 140.

At operation 504, the controller 150 adjusts the inlet valves 164a, 164b of the gas blender 160 to control the concentration of oxygen in the oxygenation gas. The concentration of oxygen in the oxygenation gas is sometimes known in the art as the “fraction of inspired oxygen”, abbreviated as “FiO2”. For example, when the first inlet 162a of the gas blender 160 is connected to a supply of medical air and the second inlet 162b of the gas blender 160 is connected to a supply of pure oxygen, the controller 150 may increase the concentration of oxygen in the oxygenation gas by closing the first inlet valve 164a and/or opening the second inlet valve 164b. Conversely, the controller 150 may decrease the concentration of oxygen in the oxygenation gas by opening the first inlet valve 164a and/or closing the second inlet valve 164b.

As part of operations 502 and 504, the controller 150 may adjust any or all of the inlet valves 164a, 164b and outlet valves 166a, 166b to control the total volumetric flow rate of oxygenation gas conveyed from the gas blender 160 to the oxygenator 140. For example, the controller 150 may increase the total volumetric flow rate of oxygenation gas conveyed from the gas blender 160 to the oxygenator 140 by opening any or all of the inlet valves 164a, 164b and outlet valves 166a, 166b. Conversely, the controller 150 may decrease the total volumetric flow rate of oxygenation gas conveyed from the gas blender 160 to the oxygenator 140 by closing any or all of the inlet valves 164a, 164b and outlet valves 166a, 166b.

It should be appreciated that operation 502 need not be performed before operation 504. Substantially the same result can be achieved by performing operation 504 before operation 502, or by performing operations 502 and 504 at the same time.

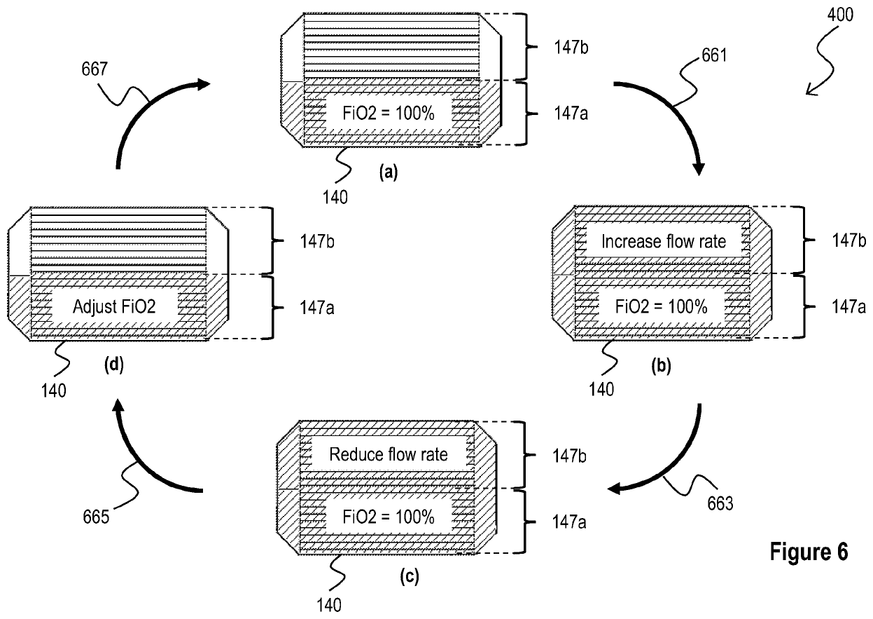

A second mode of operation of the oxygenator 140 is shown in FIG. 6. In the second mode of operation, the gas blender 160 uses the full functionality of the multi-region oxygenator 140. That is, the gas blender 160 selects whether to convey oxygenation gas to only a subset of the gas-blood interface regions 147a, 147b (e.g., to only the first gas-blood interface region 147a), or whether to convey oxygenation gas to all gas-blood interface regions 147a, 147b at the same time. The second mode of operation allows fine control over the exchange of gases within the oxygenator 140, which in turn can be used to achieve fine control over the saturation of oxygen, carbon dioxide and/or nitrogen in the patient's blood.

As shown in FIG. 6, the oxygenator 140 may have any of several “states” when operating in the second mode. In particular, FIG. 6 illustrates a series of states that may occur during treatment of a patient who experiences a low metabolic rate, inadequate carbon dioxide removal, or low oxygen saturation (e.g., cyanosis).

FIG. 6 is divided into a series of four states, (a)-(d), which will be described in sequence below. The transitions between the states are illustrated by arrows 661, 663, 665 and 667. In each state, the oxygenator 140 is depicted. The following description will begin with state (a), but the process is cyclic and so any state may be considered the “start”. In state (a), the first gas-blood interface region 147a of the oxygenator 140 is supplied with an oxygenation gas by the gas blender 160, which is indicated by the hatched lines in the first gas-blood interface region 147a. The second gas-blood interface region 147b is not supplied with oxygenation gas, which is indicated by the absence of hatched lines from the second gas-blood interface region 147b. As depicted in FIG. 6, the oxygenation gas is supplied with an FiO2 of 100%. That is, in state (a), the oxygenation gas consists solely of oxygen. The oxygenator 140 may be operated in this state in order to remove carbon dioxide from the blood of the patient, while also providing oxygen to the blood. However, given that the first gas-blood interface region 147a represents only a proportion (e.g. 40%) of the overall gas-blood interface, the supply of oxygen gas may not be sufficient to cause the patient's blood oxygen saturation to reach a desired value (referred to herein as a “target value”). The target blood oxygen saturation may be chosen by a clinician, based upon the clinical needs of a patient. A sensor 110 may measure the blood oxygen saturation, and provide the measurement to the controller 150. The controller 150 may compare the measured blood oxygen saturation with the target value and, when the measured blood oxygen saturation is below the target value, the controller 150 and gas blender 160 may cause the oxygenator 140 to transition to state (b), as indicated by arrow 661.

In state (b), the first gas-blood interface region 147a continues to be supplied with oxygenation gas at 100% FiO2. However, the second gas-blood interface region 147b now begins to be supplied with the oxygenation gas, as indicated by the hatched lines in the second gas-blood interface region 147b. That is, the flow rate of the oxygenation gas to the second gas-blood interface region 147b may be increased from zero. The flow rate of the oxygenation gas to the second gas-blood interface region 147b may continue to be increased until the target value is reached (e.g., until the sensor 110 measures a blood oxygen saturation level equal to the target value). Supplying the second gas-blood interface region 147b with the oxygenation gas increases the amount of oxygen to which the blood is exposed beyond what is possible by only supplying the first gas-blood interface region 147a. Therefore, a higher saturation of oxygen may be achieved by supplying both interface regions 147a, 147b. Both gas-blood interface regions 147a, 147b receive oxygenation gas with substantially the same composition because the oxygenation gas is mixed within the mixing chamber 161 before being conveyed to the oxygenator 140. The oxygenator may operate in state (b) to increase the saturation of oxygen in the patient's blood to a healthy level (e.g. 98.5%). However, during a surgical procedure, the patient's metabolic rate may drop. This, in turn, means that the blood needs to be exposed to a lower amount of oxygen in the oxygenator 140 to achieve the same saturation of oxygen in the patient's blood. This drop in metabolic rate may be detected in the form of an increase in the saturation of oxygen in the patient's blood caused by the continual supply of the oxygenation gas with the same oxygen concentration and with the same flow rate in spite of the drop in metabolic rate. This may be detected, for example, by the sensor 110. In such a case, the controller 150 and gas blender 160 may cause the oxygenator 140 to transition to state (c), as indicated by arrow 663.

In state (c), the flow rate of oxygenation gas to the second gas-blood interface region 147b is reduced. The amount of oxygen to which the blood is exposed is thus reduced, leading to a decrease in the patient's blood oxygen saturation. The measured blood oxygen saturation may be reduced to the target value at a lower, but non-zero, flow rate of oxygenation gas to the second gas-blood interface region 147b. Alternatively, the flow rate of the oxygenation gas to the second gas-blood interface region 147b may need to be reduced to zero (such that there is no oxygenation gas in the second gas-blood interface region 147b) before the target blood oxygen saturation is achieved. In some instances, it may be that this reduction to zero still is not a sufficient reduction to achieve the target value. Therefore, to continue to reduce the amount of oxygen to which the blood is exposed (e.g. in response to the flow rate in the second gas-blood interface region 147b being reduced to zero), the controller 150 and gas blender 160 may cause the oxygenator 140 to transition to state (d), as indicated by arrow 665.

In state (d), there is no supply of oxygenation gas to the second gas-blood interface region 147b, as indicated by the absence of hatched lines from the second gas-blood interface region 147b. To further reduce the amount of oxygen to which the blood is exposed, the concentration of oxygen in and/or the flow rate of the oxygenation gas conveyed to the first gas-blood interface region 147a may be reduced. This further reduces the amount of oxygen in the oxygenator 140 and thus allows the patient's blood oxygen saturation to be further reduced until the measured value reaches the target value. Advantageously, this allows the controller 150 to effectively manage the blood oxygen saturation of a patient who is experiencing a low metabolic rate (e.g. a patient whose body temperature has been deliberately lowered during a cardiac perfusion procedure). As the patient returns to a higher (e.g. normal) metabolic rate, the FiO2 and/or the flow rate of the oxygenation gas conveyed to the first gas-blood interface region 147a can be increased again to increase the amount of oxygen present in the oxygenator 140. The FiO2 can continue to be increased until it reaches 100%. The controller 150 and gas blender 160 thus cause the oxygenator 140 to transition back to state (a), as indicated by arrow 667.

When the controller 150 and gas blender 160 cause the oxygenator 140 to operate in accordance with the second mode, as discussed above, each of the gas-blood interface regions 147a, 147b has a different primary purpose. The primary purpose of the first gas-blood interface region 147a is to remove (or “sweep”) carbon dioxide from the blood and, therefore, the first gas-blood interface region 147a is described as a “sweep chamber”. The oxygenation gas supplied to the first gas-blood interface region 147a is described as a “sweep gas”. The primary purpose of the second gas-blood interface region 147b is to control the blood oxygen saturation and, therefore, the second gas-blood interface region 147b is described as an “oxygen regulation chamber”. The different primary purposes of the gas-blood interface regions 147a, 147b are achieved by the way in which gas blender 160 conveys oxygenation gas to the gas-blood interface regions 147a, 147b, rather than due to any fundamental differences in the structure of the gas-blood interface regions 147a, 147b themselves. It should be appreciated that the first gas-blood interface region 147a may contribute to controlling the blood oxygen saturation, and the second gas-blood interface region 147b may contribute to removing carbon dioxide from the blood, even though those are not the primary purposes of those regions. That is, the first and second gas-blood interface regions 147a, 147b control both blood oxygen saturation and the partial pressure of carbon dioxide in the blood. Adjusting the total gas flow and gas composition (FiO2) between these gas-blood interface regions affects the partial pressure of carbon dioxide in the blood as well as the blood oxygen saturation, and the controller 150 manages both the oxygen saturation and carbon dioxide pressure by automatically adjusting gas flows between the different gas-blood interface regions 147a, 147b.

FIG. 6 represents a particular example of the operation of the oxygenator 140 in a patient who initially has a normal metabolic rate and subsequently has a low metabolic rate. In practice, a patient's metabolic rate may increase or decrease during a surgical procedure and thus the oxygenator 140 may move freely between all of the states in FIG. 6. That is, the sequence of states is not restricted merely to the order described but instead may increase and/or decrease the oxygen concentration and/or the flow rate of the oxygenation gas to the first and/or second gas-blood interface regions 147a, 147b as needed to achieve the target blood oxygen saturation value.

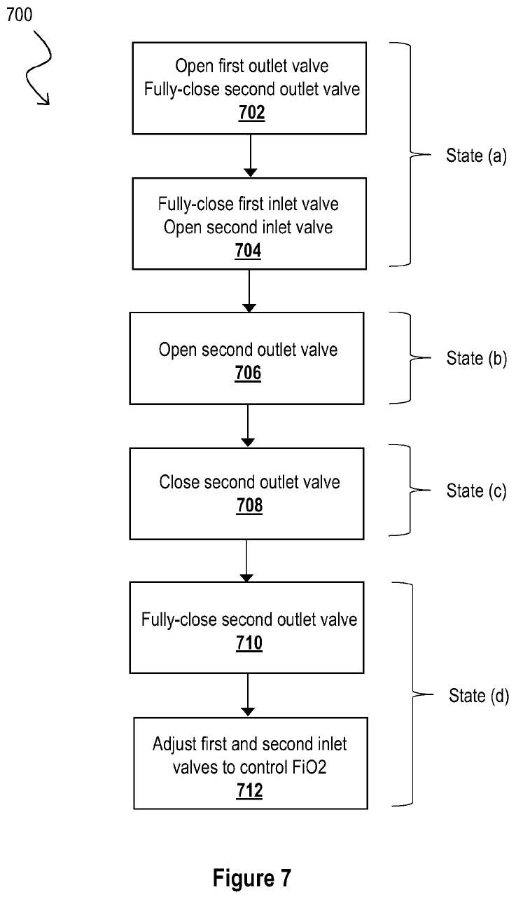

A method 700 of causing the oxygenator 140 to operate in the second mode is illustrated in FIG. 7. The method 700 may begin at operation 702, at which the controller 150 opens the first outlet valve 166a of the gas blender 160 and fully-closes the second outlet valve 166b of the gas blender 160. Opening the first outlet valve 166a allows oxygenation gas to flow from the gas blender 160 and into the first gas-blood interface region 147a of the oxygenator 140. Fully-closing the second outlet valve 166b prevents oxygenation gas flowing from the gas blender 160 and into the second gas-blood interface region 147b of the oxygenator 140.

At operation 704, the controller 150 fully-closes the first inlet valve 164a of the gas blender 160 and opens the second inlet valve 164b of the gas blender 160. When the first inlet 162a of the gas blender 160 is connected to a supply of medical air and the second inlet 162b of the gas blender 160 is connected to a supply of pure oxygen, fully-closing the first inlet valve 164a and opening the second inlet valve 164b ensures that the oxygenation gas consists only of oxygen.

While performing operations 702 and 704, the oxygenator 140 is in state (a) as shown in FIG. 6. It should be appreciated that operation 702 need not be performed before operation 704. Substantially the same result can be achieved by performing operation 704 before operation 702, or by performing operations 702 and 704 at the same time. After performing operations 702 and 704, the method 700 may proceed either to operation 706 or to operations 710 and 712.

At operation 706, the controller 150 opens the second outlet valve 166b of the gas blender 160. Opening the second outlet valve 166b allows oxygenation gas to flow from the gas blender 160 and into the second gas-blood interface region 147b of the oxygenator 140. During operation 706, the controller 150 keeps the first outlet valve 166a open, so that oxygenation gas continues to flow into the first gas-blood interface region 147a of the oxygenator 140. The controller 150 also keeps the first inlet valve 164a fully-closed, and keeps the second inlet valve 164b open, so that the oxygenation gas consists only of oxygen. While performing operation 706, the oxygenator 140 is in state (b) as shown in FIG. 6. After performing operation 706, the method 700 may proceed to operation 702, to operation 708, or to operations 710 and 712.

At operation 708, the controller 150 closes the second outlet valve 166b of the gas blender 160. Closing the second outlet valve 166b reduces the flow rate of oxygenation gas from the gas blender 160 to the second gas-blood interface region 147b of the oxygenator 140. During operation 708, the controller 150 keeps the first outlet valve 166a open, so that oxygenation gas continues to flow into the first gas-blood interface region 147a of the oxygenator 140. The controller 150 also keeps the first inlet valve 164a fully-closed, and keeps the second inlet valve 164b open, so that the oxygenation gas consists only of oxygen. While performing operation 708, the oxygenator 140 is in state (c) as shown in FIG. 6. After performing operation 708, the method 700 may proceed to operation 702, to operation 706, or to operations 710 and 712. In particular, the method 700 may alternate between operations 706 and 708, such that second outlet valve 166b is opened and closed to regulate the amount of oxygen entering the second gas-blood interface region 147b, and thereby maintain the patient's blood oxygen saturation at a desired level.

At operation 710, the controller 150 fully-closes the second outlet valve 166b of the gas blender 160. Fully-closing the second outlet valve 166b prevents oxygenation gas flowing from the gas blender 160 and into the second gas-blood interface region 147b of the oxygenator 140. During operation 710, the controller 150 keeps the first outlet valve 166a open, so that oxygenation gas continues to flow into the first gas-blood interface region 147a of the oxygenator 140.

At operation 712, the controller 150 adjusts the inlet valves 164a, 164b of the gas blender 160 to control the concentration of oxygen in the oxygenation gas. For example, when the first inlet 162a of the gas blender 160 is connected to a supply of medical air and the second inlet 162b of the gas blender 160 is connected to a supply of pure oxygen, the controller 150 may increase the concentration of oxygen in the oxygenation gas by closing the first inlet valve 164a and/or opening the second inlet valve 164b. Conversely, the controller 150 may decrease the concentration of oxygen in the oxygenation gas by opening the first inlet valve 164a and/or closing the second inlet valve 164b. Thus, at operation 712, the controller 150 opens and closes the inlet valves 164a, 164b to regulate the amount of oxygen entering the first gas-blood interface region 147a, and thereby maintain the patient's blood oxygen saturation at a desired level.

While performing operations 710 and 712, the oxygenator 140 is in state (d) as shown in FIG. 6. It should be appreciated that operation 710 need not be performed before operation 712. Substantially the same result can be achieved by performing operation 712 before operation 710, or by performing operations 710 and 712 at the same time. After performing operations 710 and 712, the method 700 typically proceeds to operation 704.

While performing operations 702 to 712, the controller 150 may adjust any of the inlet valves 164a, 164b or outlet valves 166a, 166b, as appropriate for a particular state of the oxygenator 140, to control the total volumetric flow rate of oxygenation gas conveyed from the gas blender 160 to the oxygenator 140.

As already mentioned, any of the states (a)-(d) in FIG. 6 may be treated as the starting state, depending on the particular clinical scenario. Hence, the method 700 may begin at any of operations 702 to 712, depending on the current state of the gas blender 160 and the patient's clinical requirements.

The controller 150 and gas blender 160 together allow a clinician (such as a perfusionist) to switch easily and quickly between the different modes of operation supported by a multi-region oxygenator. For example, a clinician can select a particular mode of operation via the user interface 210 (see FIG. 3), without having to manually change any of the connections between the supply gas sources 174a, 174b, the gas blender 160 and the oxygenator 140.

Furthermore, the controller 150 can cause the oxygenator 140 to transition automatically between the various states of the second operating mode, as shown in FIG. 6, without the need for a clinician to manually intervene. For example, the controller 150 may automatically adjust the inlet valves 164a, 164b and outlet valves 166a, 166b based upon a measured value of a physiological parameter indicative of the oxygenation of the patient's blood. The controller 150 may automatically adjust the inlet valves 164a, 164b and outlet valves 166a, 166b to minimise a difference between the measured value of a physiological parameter and a target value of the physiological parameter. The physiological parameter may be an arterial saturation of oxygen, abbreviated as “SaO2”, which is defined as the saturation of oxygen in the arterial blood of a patient as measured directly from the blood. The arterial saturation of oxygen may be measured by a sensor 110 positioned downstream of the oxygenator 140 (i.e., the blood flows past the sensor 110 after having passed through the oxygenator 140). Alternatively, the physiological parameter may be a peripheral saturation of oxygen, abbreviated as “SpO2”. The arterial saturation of oxygen may be measured by a pulse oximetry sensor positioned on the patient. Alternatively or in addition, the physiological parameter may be a partial pressure of carbon dioxide, abbreviated as “PaCO2”. The partial pressure of carbon dioxide may be measured by a sensor 110 positioned downstream of the oxygenator 140. Thus, the controller 150 may automatically adjust the inlet valves 164a, 164b and outlet valves 166a, 166b to maintain the patient's blood oxygen and/carbon dioxide levels at a target value.

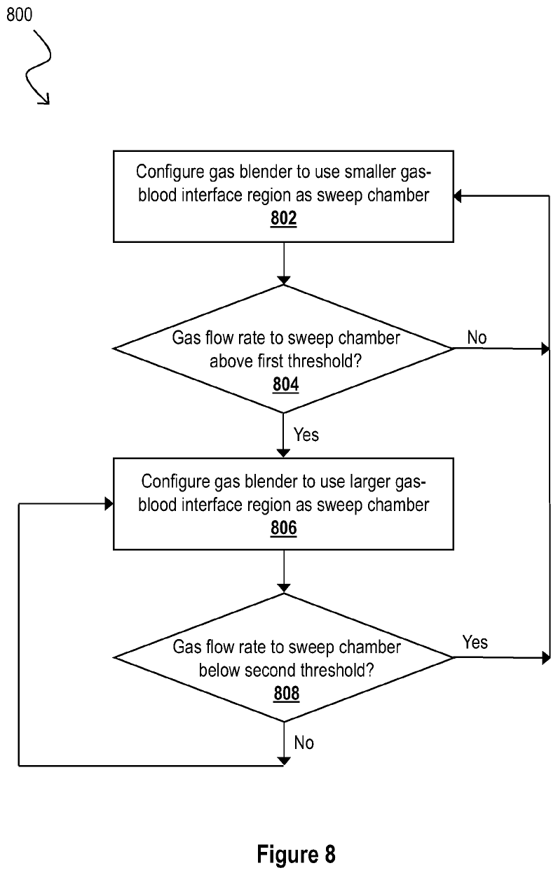

FIG. 8 is a flow chart of a method 800 in which the controller 150 can automatically switch between using different gas-blood interface regions 147a, 147b of the oxygenator 140 as a sweep chamber. As mentioned above, each gas-blood interface region 147a, 147b may have a different size (e.g., volume or surface area). For example, the first gas-blood interface region 147a may comprise 40% of the gas-blood interface 147 and the second gas-blood interface region 147b may comprise 60% of the gas-blood interface 147. Usually, the smaller gas-blood interface region 147a acts as a sweep chamber (i.e., a gas-blood interface region whose primary purpose is to remove carbon dioxide from the blood) and the larger gas-blood interface region 147b acts as an oxygen regulation chamber (i.e., a gas-blood interface region whose primary purpose is to control the saturation of oxygen in the blood). However, sometimes the smaller gas-blood interface region 147a is not large enough to remove carbon dioxide effectively from the patient's blood. For example, the smaller gas-blood interface region 147a may not have a sufficient surface area to remove an adequate amount of the carbon dioxide produced by patients with a large body mass. To ensure the efficacy of a cardiac perfusion process performed on such patients, the controller 150 can perform the method 800 shown in FIG. 8 to automatically switch to using the larger gas-blood interface region 147b as the sweep chamber.

The method 800 begins at operation 802, in which the controller 150 configures the gas blender 160 to use the smaller gas-blood interface region (i.e., the first gas-blood interface region 147a) as the sweep chamber. In more detail, the controller 150 keeps the first outlet valve 166a of the gas blender 160 open to ensure that oxygenation gas flows through the first gas-blood interface region 147a to remove carbon dioxide from the blood. The controller 150 adjusts (e.g., opens, closes and/or fully-closes) the second outlet valve 166b to control the supply of oxygenation gas to the second gas-blood interface region 147b, and thereby regulate the patient's blood oxygen saturation. Operation 802 may include performing the method 700 of FIG. 7, so as to cause the oxygenator 140 to transition between the various states shown in FIG. 6.

Whilst performing operation 802, the controller 150 monitors the gas flow rate to the sweep chamber. For example, the controller 150 may receive measurements of the volumetric flow rate made by a sensor positioned between the mixing chamber 161 and the first outlet 167a of the gas blender 160.

At operation 804, the controller 150 compares the measured gas flow rate with a first threshold. The first threshold is a predetermined value that is indicative of an “excessive” flow rate of oxygenation gas to the sweep chamber. If the measured gas flow rate is less than or equal to the first threshold, the controller 150 continues to use the smaller gas-blood interface region as the sweep chamber; the method returns to operation 802. Alternatively, if the measured gas flow rate is greater than the first threshold, it is necessary to use the larger gas-blood interface region as the sweep chamber to ensure adequate carbon dioxide removal; the method then proceeds to operation 806.

At operation 806, the controller 150 configures the gas blender 160 to use the larger gas-blood interface region (i.e., the second gas-blood interface region 147b) as the sweep chamber. To do this, the controller 150 effectively switches the roles of the first and second outlet valves 166a, 166b, such that the first outlet valve 166a acts as if it were the second outlet valve 166b when performing the method 700 of FIG. 7, and the second outlet valve 166b acts as if it were the first outlet valve 166a when performing method 700. In more detail, the controller 150 keeps the second outlet valve 166b of the gas blender 160 open to ensure that oxygenation gas flows through the second gas-blood interface region 147b to remove carbon dioxide from the blood. The controller 150 adjusts (e.g., opens, closes and/or fully-closes) the first outlet valve 166a to control the supply of oxygenation gas to the first gas-blood interface region 147a, and thereby regulate the patient's blood oxygen saturation. Operation 804 may cause the oxygenator 140 to transition between the various states shown in FIG. 6 but, rather than performing method 700 exactly as shown in FIG. 7, the roles of the first and second outlet valves 166a, 166b are switched. In this manner, the rate at which carbon dioxide is removed from the patient's blood is increased by using the larger gas-blood interface region as the sweep chamber.

Whilst performing operation 806, the controller 150 monitors the gas flow rate to the sweep chamber. For example, the controller 150 may receive measurements of the volumetric flow rate made by a sensor positioned between the mixing chamber 161 and the second outlet 167b of the gas blender 160.

At operation 808, the controller 150 compares the measured gas flow rate with a second threshold. The second threshold is a predetermined value that is indicative of a “low” flow rate of oxygenation gas to the sweep chamber. If the measured gas flow rate is less than the second threshold, the controller 150 reverts to using the smaller gas-blood interface region as the sweep chamber; the method returns to operation 802. Alternatively, if the measured gas flow rate is greater than or equal to the second threshold, the controller 150 continues to use the larger gas-blood interface region as the sweep chamber; the method then returns to operation 806.

By performing the method 800 shown in FIG. 8, the controller 150 can automatically switch between using different gas-blood interface regions 147a, 147b of the oxygenator 140 as the sweep chamber, without the need for a clinician to manually change any of the connections between the supply gas sources 174a, 174b, the gas blender 160 and the oxygenator 140.

It will be understood that the invention has been described above purely by way of example, and that modifications of detail can be made within the scope of the claims. In particular, the sequence of operations shown in FIGS. 5, 7 and 8 are merely exemplary. Any of the operations shown in methods 500, 700 and 800 may be performed in a different order that achieves substantially the same result.

1. An apparatus for controlling a supply of an oxygenation gas to an oxygenator, the apparatus comprising:
a mixing chamber having an internal volume, a plurality of inlets and a plurality of outlets, wherein each inlet is configured to receive a different supply gas, and each outlet is configured to convey an oxygenation gas from the internal volume to the oxygenator;
a plurality of inlet valves, wherein each inlet valve is disposed between a respective inlet and the internal volume of the mixing chamber;
a plurality of outlet valves, wherein each outlet valve is disposed between the internal volume of the mixing chamber and a respective outlet; and
a controller configured to open and close each of the inlet valves and each of the outlet valves independently of one another.

2. The apparatus of claim 1, wherein the plurality of inlet valves includes a first inlet valve and a second inlet valve, and wherein the controller configured to adjust the first inlet valve and the second inlet valve to control a concentration of oxygen in the oxygenation gas.

3. The apparatus of claim 1, wherein the controller is further configured to cause the oxygenator to operate in a first mode by opening all of the outlet valves.

4. The apparatus of claim 3, wherein opening all of the outlet valves comprises:
opening each outlet valve to a respective position, wherein each position is chosen to divide a volumetric flow rate of the oxygenation gas through the plurality of outlets in accordance with a predetermined ratio.

5. The apparatus of claim 4, wherein the predetermined ratio is chosen to be substantially equal to a ratio between sizes of respective gas-blood interface regions of the oxygenator.

6. The apparatus of claim 1, wherein the plurality of outlet valves includes a first outlet valve and a second outlet valve, and wherein the controller is configured to cause the oxygenator to operate in a second mode by:
adjusting the first outlet valve between a partially-open position and a fully-open position; and
adjusting the second outlet valve between a closed position and an open position.

7. The apparatus of claim 6, wherein the controller is configured to cause the oxygenator to adjust the first and second outlet valves by:
opening the first outlet valve and fully-closing the second outlet valve; and
after fully-closing the second outlet valve, opening the second outlet valve.

8. The apparatus of claim 7, wherein the controller is configured to cause the oxygenator to adjust second outlet valve by:
after opening the second outlet valve, fully-closing the second outlet valve.

9. The apparatus of claim 6, wherein the plurality of inlet valves includes a first inlet valve and a second inlet valve, and wherein the controller is further configured to cause the oxygenator to operate in the second mode by:
when the second outlet valve is closed, adjusting the first inlet valve and the second inlet valve to control a concentration of oxygen in the oxygenation gas.

10. An The apparatus of claim 6, wherein the plurality of inlet valves includes a first inlet valve and a second inlet valve, and wherein the controller is further configured to cause the oxygenator to operate in the second mode by:
maintaining the first inlet valve in a closed position while the second inlet valve is open and while the first and second outlet valves are open.

11. The apparatus of claim 10, wherein the controller is further configured to cause the oxygenator to operate in the second mode by:
adjusting the second outlet valve between a closed position and an open position while the first inlet valve is closed, while the second inlet valve is closed and while the first outlet valve is open.

12. The apparatus of claim 1, wherein:
a first inlet of the plurality of inlets is configured to be connected to a supply of medical air from a hospital gas supply or a gas cylinder;
a second inlet of the plurality of inlets is configured to be connected to a supply of oxygen from a hospital gas supply or a gas cylinder; and
each outlet is configured to be connected to a respective inlet of a multi-region oxygenator.

13-14. (canceled)

15. The apparatus of claim 1, wherein the plurality of inlets and the plurality of outlets are in fluid communication with the internal volume of the mixing chamber.

16. The apparatus of claim 1, further comprising a pressure sensor configured to measure pressure within the internal volume of the mixing chamber
wherein the controller is configured to:
receive a first pressure measurement from the pressure sensor; and
open and/or close any of the inlet valves and/or any of the outlet valves to minimise a difference between the pressure measurement and a target pressure.

17. (canceled)

18. The apparatus of claim 1, further comprising a pressure sensor configured to measure pressure within the internal volume of the mixing chamber, wherein the controller is configured to:
receive a second pressure measurement from the pressure sensor; and
close at least one of the inlet valves when the second pressure measurement exceeds a safety threshold.

19. The apparatus of claim 1, wherein the controller is further configured to control a flow rate of the oxygenation gas through at least one of the outlet valves by adjusting an inlet valve and/or adjusting the at least one outlet valve.

20. The apparatus of claim 1, wherein the mixing chamber, the plurality of inlet valves and the plurality of outlet valves are integrated within a gas blender.

21. The apparatus of claim 1, wherein the controller is further configured to:
open the first outlet valve to maintain a flow of oxygenation gas through a first outlet to a first gas-blood interface region of the oxygenator, while adjusting the second outlet valve between a fully-closed position and an open position to control a flow of oxygenation gas through a second outlet to a second gas-blood interface region of the oxygenator, wherein the first gas-blood interface region of the oxygenator is smaller than the second gas-blood interface region of the oxygenator; and
in response to determining that the flow of oxygenation gas through the first outlet exceeds a first threshold, open the second outlet valve to maintain a flow of oxygenation gas through the second outlet to the first gas-blood interface region while adjusting the first outlet valve between a fully-closed position and an open position to control a flow of oxygenation gas through the first outlet to the first gas-blood interface.

22. The apparatus of claim 21, wherein the controller is further configured to:
in response to determining that the flow of oxygenation gas through the second outlet is below a second threshold, open the first outlet valve to maintain a flow of oxygenation gas through the first outlet to the first gas-blood interface region while adjusting the second outlet valve between the fully-closed position and the open position to control the flow of oxygenation gas through the second outlet to the second gas-blood interface region.

23. A cardiac perfusion system comprising:
the apparatus of claim 1; and
an oxygenator having a plurality of gas-blood interface regions and plurality of oxygenator gas inlets, wherein each oxygenator gas inlet is configured to receive an oxygenation gas and to convey the oxygenation gas to a respective gas-blood interface regions of the plurality of gas-blood interface regions,
wherein each outlet of the mixing chamber is connected to a respective oxygenator gas inlet.

24. (canceled)
